(19) United States
(12) Patent Application Publication
(10) Pub. No.: US 2021/0299298 A1
(43) Pub. Date: Sep. 30, 2021

(54) PORTABLE STERILIZATION DEVICE

(71) Applicant: Acera LLC, Beverly, MA (US)

(72) Inventors: Thomas V. Root, Beverly, MA (US); Michael Cook, Salem, MA (US); Michael S. Epstein, Annapolis, MD (US)

(21) Appl. No.: 17/205,529

(22) Filed: Mar. 18, 2021

Related U.S. Application Data
(60) Provisional application No. 63/000,891, filed on Mar. 27, 2020.
(60) Provisional application No. 62/994,600, filed on Mar. 25, 2020.

(51) Int. Cl. A61L 2/10

(52) U.S. Cl. CPC A61L 2/10; A61L 2202/11; A61L 2202/16

(57) ABSTRACT

In one aspect, a portable sterilization device configured to be carried by an individual is disclosed, which comprises a wearable element configured for removable and replaceable coupling to a body part of the individual, an ultraviolet light emitting diode (UV LED) coupled to said element and positioned so as to emit radiation into an external environment, and a portable power supply for supplying electrical power to said UV LED.

### RELATED APPLICATION

The present application claims priority to Provisional Application No. 62/994,600 filed on Mar. 25, 2020 and Provisional Application No. 63/000,891 filed on Mar. 27, 2020 having the same titles as the present application which are herein incorporated by reference in its entirety.

### BACKGROUND

The present disclosure relates generally to sterilization devices, and more particularly to portable sterilization devices that can be carried by an individual by removably and replaceably coupling it to a body part of the individual.

With the outbreak of the COVID-19 virus, the need for sterilization of various surfaces is becoming more acute. Many sterilization devices are bulky, expensive and cumbersome to use.

Accordingly, there is a need for sterilization devices that can be easily employed and carried from one place to another.

### SUMMARY

In one aspect, a portable sterilization device configured to be carried by an individual is disclosed, which comprises a wearable element configured for removable and replaceable coupling to a body part of the individual, an ultraviolet light emitting diode (UV LED) coupled to said element and positioned so as to emit radiation into an external environment, and a portable power supply for supplying electrical power to the UV LED.

In some embodiments, the portable sterilization device comprises a coupling member for removably and replaceably securing the portable power supply to a body part of the individual. In some embodiments, the portable power supply can be one or more batteries, e.g., rechargeable Li-ion batteries, which can, for example, be coupled in series to supply the requisite voltage to the LED.

In some embodiments, the coupling member can be a wrist strap that can be used to secure the portable power supply to an individual's wrist.

In some embodiments, the UV LED emits radiation with a wavelength in a range of about 200 nm to about 280 nm, which can provide germicidal effects. By way of example, in some embodiments, the UV radiation can have a wavelength of about 222 nm. In some embodiments, an LED employed in the practice of the present teachings can emit radiation at a power of about 30 mW. In some such embodiments, a surface can be exposed to the LED radiation at a distance of less than about 6 inches and for a time duration of at least about 10 seconds to sterilize the surface. In some embodiments, commercially available UVC LEDs emitting radiation with a wavelength of 265 nm or 275 nm can be employed. By way of example, such an LED can be obtained from Luminus of Sunnyvale, Calif. under the trade designation XST-3525-UV. This LED can generate UV radiation at a wavelength of 275 nm with a 60-degree view angle and can be mounted onto a surface, such as a surface of a ring or a finger cot as disclosed herein.

In some embodiments, the portable sterilization device can further include a switch for electrically coupling and decoupling the UV LED to the portable power supply.

In some embodiments, the wearable element includes a finger cot that is configured to receive, at least partially, a digit of the individual. In some embodiments, the UV LED is coupled to the finger cot so as to be positioned in proximity to a tip of the digit to which the finger cot is coupled.

In some embodiments, the wearable element includes a metallic thimble having an inner electrically insulating layer to protect the individual's finger when inserted into the thimble. In some such embodiments, an LED, and its associated printed circuit board (PCB), can be mounted on the metallic thimble, where the metallic thimble can act as a heat sink.

In some embodiments, the wearable element incudes a ring configured to be worn by an individual, and a UV LED is mounted onto an outer surface of the ring.

In some embodiments, an electrical conduit (e.g., a twisted pair of copper wires) electrically couples the portable power supply to the UV LED to supply electrical power thereto. In some embodiments, a wrist strap is employed to secure at least a portion of the electrical conduit to the individual wrist.

In some embodiments, the wearable element includes a bracelet onto an external surface of which one or more UV LEDs, such as those disclosed herein, can be mounted.

In a related aspect, a portable sterilization device configured to be carried by an individual is disclosed, which includes a UV LED radiation source, and a portable power supply that is electrically connected to the UV LED radiation source to provide electrical power thereto. The device can further include a radiation waveguide that extends from a proximal end to a distal end, where the radiation waveguide is optically coupled at its proximal end to the UV LED radiation source to receive radiation therefrom. The radiation waveguide guides the received radiation from its proximal end to its distal end through which the radiation exits the waveguide. The portable sterilization device can further include a wearable element to which a section of said radiation waveguide is secured. The device can further include a coupling element for removably and replaceably coupling the portable power supply to a body portion of the individual. By way of example, the portable power supply can be removably and replaceably coupled to the individual's arm via an arm cuff.

In some embodiments, the wearable element can be a finger ring to a surface of which a section of the radiation waveguide (e.g., a section in proximity of its distal end) can be attached, e.g., via gluing or otherwise.

Further understanding of various aspects of the present invention can obtained by reference to the following detailed description in conjunction with the associated drawings, which are described briefly below.

### BRIEF DESCRIPTION OF THE DRAWINGS

FIG. 1 schematically depicts a portable sterilization device, which includes a finger cot to which a UV LED device is mounted and which can be easily carried by an individual and can be effectively utilized for sterilization of various surfaces,

FIG. 2 schematically depicts a portable sterilization device according to another embodiment in which a UV LED is mounted onto an outer surface of a ring that can be worn by an individual,

FIG. 3 schematically depicts a portable sterilization device according to yet another embodiment in which an UV LED is mounted onto an outer surface of a bracelet, which can be worn by an individual, and

FIG. 4 schematically depicts a portable sterilization device according to another embodiment in which a radiation waveguide is coupled at a proximal end to a UV LED and through its distal end radiation is transmitted into the external environment.

### DETAILED DESCRIPTION

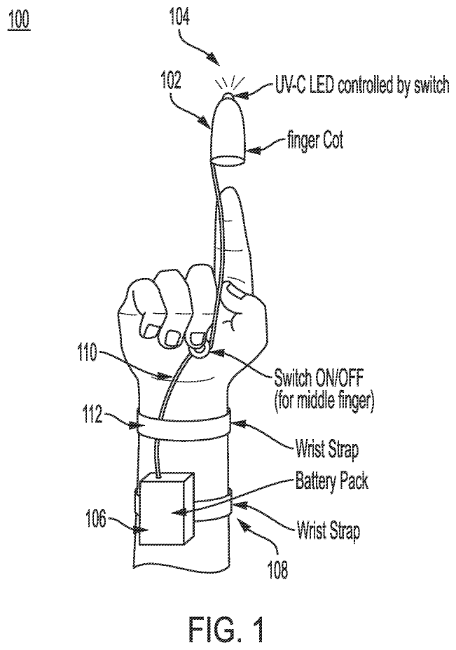

FIG. 1 schematically depicts a portable sterilization device 100 according to an embodiment, which includes a wearable element 102 into which an individual's finger, such as the individual's index finger, can be at least partially, and removably, inserted. An ultra-violet (UV) radiation source 104, which is in the form of a light-emitting diode (LED) in this embodiment, is coupled to the finger cot and is oriented so as to emit radiation into the external environment. In some embodiments, the wearable element 102 can be a finger cot. Alternatively, the wearable element 102 can be a metallic thimble having an electrically insulating inner layer for protecting a user's finger.

The UV LED 104 can be coupled to the wearable element in a variety of different ways. By way of example, the UV LED 104 can be glued to an outer surface of the wearable element. Alternatively, the UV LED 104 and its associated printed circuit board (PCB) can be coupled to an outer surface of metallic thimble such that the metallic thimble would act as a heat sink for removing heat from the LED element.

In some embodiments, the UV LED 104 emits UV radiation with a wavelength in a range of about 200 nm to about 280 nm. Further, in this embodiment, the UV LED 104 can emit radiation at a power of about 30 mW, though other emission powers can also be used.

The portable sterilization device 100 further includes a portable power supply 106 that can be removably coupled to the individual's wrist via a wrist strap 108. An electrical conduit 110, e.g., a twisted pair of copper wires, electrically connects the portable power supply 106 to the UV LED 104 to provide electrical power thereto. In this embodiment, a wrist strap 112 allows securing a portion of the electrical conduit 110 to the individual's wrist.

Once the wearable element (e.g., a finger cot or a thimble) is coupled to the individual's finger, the individual can use the radiation emitted by the UV LED to sterilize various surfaces in an efficient manner, e.g., by simply scanning the finger tip across a surface.

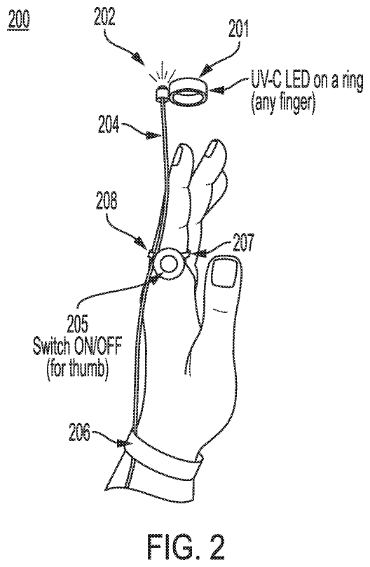

FIG. 2 schematically depicts a portable sterilization device 200 according to another embodiment, which includes a ring 201 that can be worn by an individual. A UV LED source 202 is mounted onto an outer surface of the ring 201. In this embodiment, the UV LED source 202 is oriented such that a central ray of a set of diverging rays emanating from the UV LED source is substantially parallel to the outer surface of the ring to which the UV LED source is mounted. In other embodiments, the UV LED source 202 can be mounted onto the outer surface of the ring 201 such that a central ray associated with a set of diverging rays emanating from the UV LED source is substantially orthogonal to the outer surface of the ring to which the UV LED source is mounted.

The UV LED source 202 can provide radiation having a wavelength and an emission power in ranges similar to those disclosed herein in connection with the previous embodiment.

Further, similar to the previous embodiment, the portable sterilization device 200 includes a portable power supply (not shown in this figure) that can be coupled to the individual's wrist via a wrist strap, similar to the previous embodiment. Further, similar to the previous embodiment, an electrical conduit 204, e.g., a twisted pair of copper wires, couples the portable power source to the UV LED to supply power thereto. Another wrist strap 206 can be employed to secure a portion of the electrical conduit to the individual's wrist to ensure that the electrical conduit is tightly retained in place.

An electrical switch 205 disposed between the portable power supply and the UV LED allows activating and deactivating the UV LED. In this embodiment, the electrical switch 205 can be secured to the individual's finger (e.g., the same finger inserted into the finger cot), e.g., via a circular strap 207. In this embodiment, both the ring 201 and the electrical switch 205 are coupled to the individual's index finger and the electrical switch is oriented such that the individual can use her thumb to activate or deactivate the switch.

Similar to the previous embodiments, the individual can use the UV LED to efficiently and easily sterilize various surfaces, e.g., by scanning the UV LED over the surface.

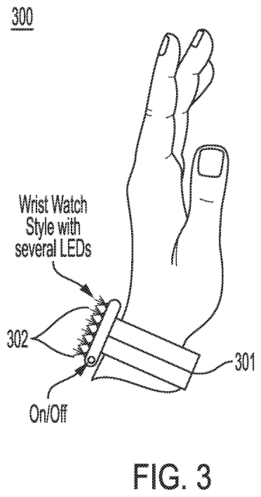

FIG. 3 schematically depicts yet another embodiment 300 of a portable sterilization device that includes a wearable element 301 in the form of a bracelet that can be worn by a user. In this embodiment, a plurality of UV LED sources 302, similar to those described above, are mounted onto an outer surface of the bracelet and are oriented so as to emit radiation into the external environment. In this embodiment, the device 300 includes a portable power supply 304, which can be removably attached via a strap 308 to the individual's arm and be coupled to the UV LED source 302 to supply electrical power thereto.

In some embodiments, the bracelet can be formed of an electrically conductive material (e.g., copper), which can act a heat sink for removing heat from an LED mounted thereto. The present teachings are not limited to the use of copper, and other electrically conductive materials can also be employed, such as gold.

In some embodiments of the present teachings, UV-C LEDs marketed by Bolb, Inc. of California, U.S.A. under the trade name (GLED) can be used.

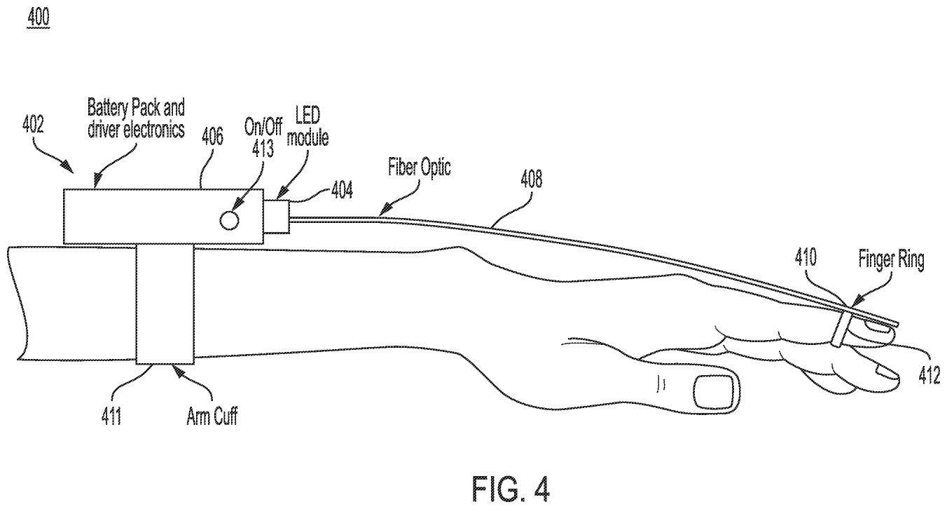

FIG. 4 schematically depicts a portable stabilization device 400 according to another embodiment having a UV light module 402, which includes a UV LED radiation source 404, such as those discussed above, and a portable power supply 406, which in this embodiment is a rechargeable battery pack, that is electrically connected to the UV LED radiation source 404 to provide electrical power thereto. In this embodiment, the battery pack 404 is releasably and replaceably attached via an arm cuff 411 to the arm of an individual carrying the portable stabilization device 400. The UV LED can emit radiation at wavelengths and at power levels disclosed above in connection with the other embodiments, For example, in some embodiments, the UV LED can emit radiation with a wavelength in a range of about 200 nm to about 280 nm. A switch 413 can be used to activate or deactivate the UV LED radiation source.

A radiation guide 408 extends from a proximal end that is optically coupled to the UV LED radiation source to a distal end through which the radiation exits the radiation guide 408 to illuminate a target of interest, e.g., a surface to be sterilized. In this embodiment, a section 410 of the radiation guide 408 is secured to an outside surface of a finger ring 412, which can be worn by the individual wearing the portable device so as to fixate the distal end over the ring finger and allow its facile use for sterilizing a surface, e.g., via scanning the distal tip of the radiation guide over a surface.

The radiation guide can be in the form of a single mode or multi-mode optical fiber. In some embodiments, the radiation waveguide can be in the form of a fiber bundle. In some implementations, the advantage of using a fiber bundle, rather than a single fiber, is that a fiber bundle can exhibit more flexibility than a single fiber, though a single fiber can also be employed in the practice of the present teachings.

The radiation guide 408 can be formed of a variety of different materials. For example, the radiation guide 408 can be formed of glass, silica, plastic, quartz or could be in the form of a liquid optical fiber. Further, in some embodiments, the radiation guide 408 can have a cross-sectional diameter, e.g., in a range of about 0.5 mm to about 4 mm, such as in a range of about 1 mm to about 2 mm.

In some embodiments, the LED output beam can have a “view angle” of about 60 degrees. By way of example, in some such embodiments, the radiation waveguide can be formed of glass (which can have an acceptance angle of about 82 degrees) or PMMA (poly methyl methacrylate) plastic (which can have an acceptance angle of about 60 degrees). This allows coupling the LED radiation into the radiation waveguide without a need for a lens, though in some other embodiments, a lens can be employed for coupling the UV radiation from the UV LED into the radiation waveguide.

In some embodiments, a jacket (not shown in the figure) can cover the radiation waveguide to prevent the leakage of the UV radiation from the lateral surface of the fiber (or a fiber bundle) into the external environment to ensure safety of the user or other individuals, especially when radiation wavelengths in a range of about 265 to 280 nm are employed. In addition, a protective eyewear can be used that filter UV-C radiation wavelengths for safety of the user. Further, as noted above, in some embodiments, UV radiation wavelengths of 222 nm or shorter may be employed to minimize the safety risks when using the portable sterilization device.

In some embodiments, the UV LED source is activated intermittently, e.g., during 5-60 second intervals. In such embodiments, passive (convection) cooling of the LED can be used. In other embodiments in which the UV LED source is used for continuous emission of radiation, active cooling can be employed, e.g., via a Peltier cooling module.

Those having ordinary skill in the art will appreciate that various changes can be made to the above embodiments without departing from the scope of the invention.

What is claimed is:

1. A portable sterilization device configured to be carried by an individual, comprising:
a wearable element configured for removable and replaceable coupling to a body part of the individual,
an ultraviolet light emitting diode (UV LED) coupled to said element and positioned so as to emit radiation into an external environment,
a portable power supply for supplying electrical power to said UV LED.

2. The portable sterilization device of claim 1, further comprising a coupling member for removably and replaceably securing said portable power supply to a body part of the individual.

3. The portable sterilization device of claim 1, wherein said UV LED emits radiation with a wavelength in a range of about 200 nm to about 280 nm.

4. The portable sterilization device of claim 1, further comprising a switch for electrically coupling and decoupling of said UV LED to said portable power supply.

5. The portable sterilization device of claim 1, wherein said wearable element comprises a finger cot configured to receive at least partially a digit of the individual.

6. The portable sterilization device of claim 1, wherein said wearable element comprises a thimble having an electrically insulating liner.

7. The portable sterilization device of claim 5, wherein said UV LED is coupled to said finger cot so as to be positioned in proximity to a tip of said digit to which the finger cot is coupled.

8. The portable sterilization device of claim 1, wherein said wearable element comprises a ring configured to be worn by the individual.

9. The portable sterilization device of claim 8, wherein said UV LED is positioned on an external surface of said ring.

10. The portable sterilization device of claim 1, wherein said coupling member comprises a wrist strap for securing said portable power supply to the individual's wrist.

11. The portable sterilization device of claim 1, further comprising at least one electrical conduit electrically coupling said portable power supply to said UV LED.

12. The portable sterilization device of claim 1, further comprising a wrist strap for securing at least a portion of said wire to the individual's wrist.

13. The portable sterilization device of claim 1, wherein said element comprises a bracelet and said UV LED is coupled to an external surface of said bracelet.

14. The portable sterilization device of claim 13, wherein said bracelet comprises an electrically conductive material.

15. The portable sterilization device of claim 14, wherein said electrically conductive material comprises copper.

16. A portable sterilization device configured to be carried by an individual, comprising:
a UV LED radiation source,
a portable power supply electrically connected to the UV LED radiation source to provide electrical power thereto,
a radiation waveguide extending from a proximal end to a distal end, said radiation waveguide being optically coupled at its proximal end to said UV LED radiation source to receive radiation therefrom, wherein said radiation waveguide guides said received radiation from said proximal end to said distal end through which the radiation exits the waveguide, and
a wearable element to which a section of said radiation waveguide is secured.

17. The portable sterilization device of claim 16, further comprising a coupling element for removably and replaceably coupling said portable power supply to a body portion of said individual.

18. The portable sterilization device of claim 17, wherein said body portion comprises the individual's arm.

19. The portable sterilization device of claim 16, wherein said wearable element comprises a finger ring.

20. The portable sterilization device of claim 19, wherein said section of the radiation waveguide is attached to a surface portion of said finger ring.

21. The portable sterilization device of claim 16, wherein UV LED emits radiation with a wavelength in a range of about 200 nm to about 280 nm,

22. The portable sterilization device of claim 16, wherein said radiation waveguide comprises an optical fiber.

23. The portable sterilization device of claim 16, wherein said radiation waveguide comprises a bundle of optical fibers.
